(19) United States
(12) Patent Application Publication
(10) Pub. No.: US 2023/0021429 A1
(43) Pub. Date: Jan. 26, 2023

(54) BASE-LABILE PROTECTING GROUPS FOR STEPWISE POLYMER SYNTHESIS

(71) Applicant: Shiyue Fang, Houghton, MI (US)

(72) Inventor: Shiyue Fang, Houghton, MI (US)

(21) Appl. No.: 17/844,023

(22) Filed: Jun. 19, 2022

Related U.S. Application Data
(60) Provisional application No. 63/220,895, filed on Jul. 12, 2021.

(51) Int. Cl. C08G 65/00; C07C 309/73; C07C 309/68; C07C 43/12

(52) U.S. Cl. CPC C08G 65/00; C07C 309/73; C07C 309/68; C07C 43/126

(57) ABSTRACT

This invention relates to the use of base-labile protecting groups for stepwise synthesis of polymers including oligomers. One or more monomers that have a base-labile protecting group at one end and a leaving group at the other are used in synthetic cycles comprising deprotection under stronger basic conditions to remove the base-labile protecting group, and coupling with a monomer under weaker basic conditions to elongate the polymer without premature deprotection of the base-labile protecting group. Advantages of the invention include the possibility to shorten the synthetic cycle from three steps in prior art methods to two steps, more efficient deprotection, more efficient coupling, and the use of less harmful and less expensive chemicals. One of the goals for stepwise polymer synthesis is to prepare monodisperse and sequence-defined polymers.

### CROSS-REFERENCE TO RELATED APPLICATIONS

This application for patent claims priority to Provisional application Ser. No. 63/220,895 filed on Jul. 12, 2021.

### FEDERALLY SPONSORED RESEARCH

This invention was made with United States government support under grant Nos. 1918585 and 1754235 awarded by the National Science Foundation. The government has certain rights in the invention.

### BACKGROUND OF THE INVENTION

The present invention relates to the field of stepwise synthesis of polymers (also called oligomers when the chain length is short). Polymers such as polyethylene glycol, polyethylene and polystyrene are usually synthesized by polymerization of one or more monomers. The process is random, and the products are complex polydisperse admixtures of molecules that have different molecular weights and different chain lengths. If more than one monomer is used, the locations of a particular monomer in the polymer are random. If the monomer or monomers contain functional groups, the locations of the functional groups are random [Badi et al 2009 Chem Soc Rev 38:3383 doi:10.1039/B806413J].

For many applications such as linking in organic synthesis and bioconjugation, drug tagging to increase solubility and stability and to reduce toxicity, nanomedicine and digital data storage, sequence-defined polymers are required or preferred [Herzberger et al 2016 Chem Rev 116:2170 doi:10.1021/acs.chemrev.5b00441, Giorgi et al 2014 Beilstein J Org Chem 10:1433 doi:10.3762/bjoc.10.147, Abd Ellah et al 2019 Nanomedicine 14:1471 doi:10.2217/nnm-2018-0348]. Sequence-defined polymers are intended to be homogeneous materials, which means that each molecule in the material has the same molecular weight and length, and if the polymer has more than one type of monomer units or different functional groups, the locations of the monomer units and functional groups are defined in the polymer [Solleder et al 2017 Macromol Rapid Comm 38:1600711 doi:10.1002/marc.201600711]. Sequence-defined polymers cannot be synthesized by polymerization due to the inherent nature of randomness of polymerization methods. Instead, they are synthesized by stepwise addition of monomers.

One type of most widely used monomers for the synthesis of sequence-define polymers comprise a protecting group at one end of a molecule and a leaving group at the other end (S001, abbreviation of “structure 001”, all molecular structures including general formulas for a class of molecules in this document are labeled by the letter S followed by a three-digit number or by a three-digit number with a lower-case letter such as S001a; for molecular structures or formulas in the claims, a different numbering format is used). The stepwise synthesis is achieved by repeating a synthetic cycle (FIG. 1). The synthetic cycle includes three steps. (1) Deprotection. An example is the conversion of S004 to S005, during which the protecting group (PG) is removed and the heteroatom X is protonated. (2) Deprotonation. An example is the conversion of S005 to S006, during which the proton on the X is removed by a base. (3) Coupling. An example is the conversion of S006 to S007, during which a new bond is formed between S006 and the monomer S001 via an S_{N}2 reaction, and the polymer grows longer. The cycle is repeated until the desired length of the polymer is achieved (FIG. 1).

The synthetic route shown in FIG. 1 is termed unidirectional iterative coupling [French et al 2009 Angew Chem Int Ed 48:1248 doi:10.1002/anie.200804623]. This route is most useful in solid phase stepwise synthesis, where R₁ is a solid support [Khanal et al 2017 Chem Eur J 23:15133 doi:10.1002/chem.201703004], and stepwise synthesis using a tag or handle to assist product purification, where R₁ is the handle or tag [Szekely et al 2014 Chem Eur J 20:10038 doi:10.1002/chem.201402186, Wawro et al 2016 Org Chem Front 3:1524 doi:10.1039/c6qo00398b]. Besides the unidirectional iterative coupling route, the monomer S001 can also be used in other routes for stepwise polymers synthesis, which include bidirectional iterative coupling, chain doubling, and chain tripling [French et al 2009 Angew Chem Int Ed 48:1248 doi:10.1002/anie.200804623]. Using these routes, the polymer can reach desired length in fewer steps. However, some of these routes give symmetrical polymers, of which the two ends of the polymer chain are identical. This is not ideal for applications where asymmetric polymers, of which the two ends of the molecules are different, are needed.

Besides biopolymers such as DNA and peptides [Fang et al 2010 Org Lett 12:3720 doi:10.1021/01101316g, Zhang et al 2014 Org Lett 16:1290 doi:10.1021/ol403426u], one of the polymers that received most attention for stepwise synthesis is polyethylene glycol (PEG) [Szekely et al 2014 Chem Eur J 20:10038 doi:10.1002/chem.201402186]. In addition, polypropylene glycol (PPG) and polytetrahydrofuran (PTHF) and many other polymers that are traditionally synthesized by polymerization may be desirable for stepwise synthesis [Andersen 1994 J Am Coll Toxicol 13:437 doi 10.3109/10915819409141005, Shimomura et al 2005 Tetrahedron 61:12160 doi:10.1016/j.tet.2005.08.121]. Recently, oligosulfides and oligosulfoxides, which are potentially useful in medicine, have also been synthesized in a stepwise manner [Halami et al 2019 Tetrahedron Lett 60:151306 doi:10.1016/j.tetlet.2019.151306]. Monomer S001 can be used for stepwise synthesis of these and many other polymers including those that contain certain functional groups. An specific example for PEG synthesis, which is the version of the route in FIG. 1 in the context of PEG synthesis, is illustrated in FIG. 2. In the example, the acid-labile protecting group 4,4′-dimethoxytrityl (DMTr) group is used [Khanal et al 2017 Chem Eur J 23:15133 doi:10.1002/chem.201703004]. However, other protecting groups such as silyl and benzyl groups have also been used for stepwise PEG synthesis [Thomas et al 2011 Tetrahedron Lett 52:4316 doi:10.1016/j.tetlet.2011.06.042, Kawasaki et al 2016 Chem Asian J 11:1028 doi:10.1002/asia.201501381].

### BRIEF SUMMARY OF THE INVENTION

This invention relates to the use of base-labile protecting groups (bIPGs), which can be deprotected by treating with one or more bases via an elimination reaction, for stepwise polymer synthesis. Many bIPGs can be used. Details will be given in the following sections. For simplicity of description here, which must not be used to limit the scope of the invention, six embodiments are given in FIG. 3. In S017a-f, RO (R is an alkyl or substituted alkyl group, and O is oxygen) stands for the molecule to be protected and the rest of the formulas is the protecting group. For example, the protecting group in S017a is the phenethyl group. Deprotection of the protecting groups is achieved by treating with a base via 1,2-elimination or 1,4-elimination as shown in FIG. 3 [Margot et al 1990 Tetrahedron 46:2425 doi:10.1016/50040-4020(01)82023-8, Margot et al 1990 Tetrahedron 46:2411 doi:10.1016/50040-4020(01)82022-6]. The deprotected product is an alkoxide in these cases, but there are other cases. The deprotection side products are S018a-f.

For simplicity of description, which must not be used to limit the scope of the invention, one embodiment of using base-labile protecting groups for stepwise polymer synthesis using the unidirectional iterative coupling approach is shown in FIG. 4. More details for embodiments using other approaches including but not limited to bidirectional iterative coupling, chain doubling, and chain tripling will be given in the Detailed Description section. S019 represents a general formula of the monomer for the stepwise synthesis. It contains a bIPG at one end and a LG at the other. The synthetic cycle, which elongates the polymer by one unit defined by the monomer (not necessarily the smallest repeating unit of the polymer), contains two steps—deprotection and coupling. An example of the deprotection step is the conversion of S022 to S023, during which S022 is treated with a base such as n-butyllithium (nBuLi), lithium diisopropylamide (LDA), potassium hexamethyldisilazide (KHMDS) or tBuOK to give the product S023. An example of the coupling step is the conversion of S023 to S024, during which S023 is reacted with S019 via an S_{N}2 reaction to give S024. Because S023 is anionic, there is no need of a deprotonation step as in the prior art cases shown in FIG. 1, and the coupling reaction can be carried out directly on the deprotection product (FIG. 3). The synthetic cycle is repeated to give the polymer with desired length. Alternatively, the intermediates after deprotection such as S023 can be isolated in the protonated form and purified. In this case, an additional step to deprotonate the protonated intermediate before the coupling step as in the cases using other protecting groups in the prior art is needed. The current invention does not exclude those embodiments because as described later, the use of bIPGs for stepwise polymer synthesis has other advantages over the methods in the prior art in addition to shortening the synthetic cycle from three steps to two steps.

For the monomer S019 to be useful for stepwise polymer synthesis, and for the process shown in FIG. 4 (and other processes using S019) to work, the bIPG in the monomer S019 must meet two criteria. Criterion (i), it must be deprotectable by a base (a stronger base) in the deprotection step. Criterion (ii), it must be stable under the basic conditions (weaker basic conditions) in the coupling step.

For simplicity of description, which must not be used to limit the scope of the invention, an embodiment concerning stepwise PEG synthesis is shown in FIG. 5. The synthetic cycle contains two steps—deprotection and coupling. An example for the deprotection step is the conversion of S027 to S028, during which the phenethyl group in S027 is removed by the base KHDMS via 1,2-elimination to give the anionic S028. An example for the coupling step is the conversing of S028 to S029, during which S028 is reacted with monomer S025 to give S029. The cycle repeats until the desired length of the polymer is reached. The target molecules S031a-c are then cleaved from the solid support using pure TFA.

### ADVANTAGES

Shortening the synthetic cycle from three steps to two steps: In the prior art, all methods use non-base-labile protecting groups. The deprotection product is in protonated form. Therefore a deprotonation step is required before each coupling step. As a result, a total of three steps are required for each synthetic cycle (see FIGS. 1 and 2) [French et al 2009 Angew Chem Int Ed 48:1248 doi:10.1002/anie.200804623, Khanal et al 2017 Chem Eur J 23:15133 doi:10.1002/chem.201703004, Szekely et al 2014 Chem Eur J 20:10038 doi:10.1002/chem.201402186, Wawro et al 2016 Org Chem Front 3:1524 doi:10.1039/c6qo00398b]. In contrast, using bIPG, the deprotection product is anionic. Thus, there is no need of a deprotonation step before each coupling step. As a results, only two steps are needed for each synthetic cycle (see FIGS. 4 and 5). Shortening the synthetic cycle is attractive to stepwise polymer synthesis because in most embodiments, the cycle needs to be repeated multiple times to reach the desired product. In addition, some polymers such as PEGs and their derivatives are used in large quantities in many fields. Shortening the synthetic cycle will save significant amounts of time, materials and labor for the production of these materials. In addition, shortening the synthetic cycle will also reduce the use of harmful materials such as solvents for product and intermediate purification, which is beneficial to the environment. Despite the advantage of shortening the synthetic cycle, the invention does not exclude the embodiments involving purifying the intermediate in its protonated form, and performing a separate deprotonation step before the coupling steps.

More efficient deprotection: Compared with the use of acid-labile protecting groups such as the widely used DMTr group [Khanal et al 2017 Chem Eur J 23:15133 doi:10.1002/chem.201703004, Szekely et al 2014 Chem Eur J 20:10038 doi:10.1002/chem.201402186, Wawro et al 2016 Org Chem Front 3:1524 doi:10.1039/c6qo00398b], using some embodiments of the invention including but not limited to using the protecting groups in S017a-d, the deprotection is much faster. The reason is that for DMTr, the deprotection is reversible, while in the case of S017a-d, the deprotection is irreversible.

More efficient coupling: Compare with the widely used DMTr group [Szekely et al 2014 Chem Eur J 20:10038 doi:10.1002/chem.201402186], most of the bIPGs are less hydrophobic, and less likely to form aggregates in polar organic solvents. Because aggregation can slow down organic reactions, when the smaller bIPGs are used, the coupling reaction can be more efficient.

Less expensive: Compared with the widely used silyl protecting groups, the use of some of the bIPGs is cheaper. The materials for installing the silyl groups and for their deprotection are expensive [Kawasaki et al 2016 Chem Asian J 11:1028 doi:10.1002/asia.201501381]. The protecting groups including but not limited to that in S017d of some embodiments of the invention are inexpensive to install and deprotect.

Less toxic: Compared with the widely used benzyl protecting group, which requires toxic palladium for deprotection [Thomas et al 2011 Tetrahedron Lett 52:4316 doi:10.1016/j.tetlet.2011.06.042], and the silyl protecting group, which is usually deprotected with reagents containing fluoride [Kawasaki et al 2016 Chem Asian J 11:1028 doi:10.1002/asia.201501381], the bIPGs are deprotected with reagents that are less environmentally harmful.

### Definitions

S,S′-(Alkane-diyl) groups are those with the formula —S-alkane-S—.

Alkenyl group is a group of carbon and hydrogen atoms containing at least one carbon-carbon double bond, from the carbon of which (the double bond) the group can be attached to another group of atoms.

N-Alkyl pyrrolyl group is a group of atoms resulted from the removal of a hydrogen atom from the ring of the N-alkyl pyrrole.

Alkynyl group is a group of carbon and hydrogen atoms containing at least one carbon-carbon triple bond, from the carbon of which (the triple bond) the group can be attached to another group of atoms.

Base-labile protecting group is a protecting group that can be deprotected by treating with a base. In this application, it is limited to those that can be deprotected under stronger basic conditions in the deprotection step in stepwise polymer synthesis but stable under the weaker basic conditions used in the coupling step of stepwise polymer synthesis.

Bis(alkylthio) groups are two —SR groups attached to the same carbon of a parent compound, where R are alkyl or substituted alkyl groups.

Carboxylate group is a group of atoms containing one carbon and two oxygen atoms having one carbon-oxygen double bonds and one carbon-oxygen single bond and a negative charge. The group can be attached to other group of atoms via the carbon atom.

Electron withdrawing group is an atom or group of atoms that draws electron density from neighboring atoms towards itself.

Furyl group is a group of atoms resulted from the removal of a hydrogen atom from the furan ring.

Leaving group is an atom or group of atoms that are able to detach with a lone pair of electrons from the electrophilic reaction partner of an S_{N}2 reaction. It includes but not limited to halides and sulfonates.

Phenyl group is a group of atoms resulted from the removal of a hydrogen atom from the benzene ring. It is abbreviated as Ph.

Polymer, in this application, refers to a linear molecule containing at least six repeating units installed by one or more monomers. The polymer may have other structural features such as branches, stars and cycles. For stepwise polymer synthesis, each monomer may install one or more of the repeating units. When polymer is short, it may also be called oligomer. In this application, oligomer and polymer are collectively called polymer.

Sulfonato group is a group of atoms containing one sulfur and three oxygen atoms having two sulfur-oxygen double bonds and one sulfur-oxygen single bond and a negative charge. The group can be attached to other group of atoms via the sulfur atom.

The words “and” and “or” in this application may be interchangeable or indicate both.

### BRIEF DESCRIPTION OF DRAWINGS

FIG. 1. Known monomers and a process for stepwise polymer synthesis.

FIG. 2. An example of using known monomers for stepwise PEG synthesis.

FIG. 3. Examples of bIPGs and the mechanism for their deprotection.

FIG. 4. Stepwise polymer synthesis using a bIPG—a unidirectional iterative coupling approach.

FIG. 5. Stepwise PEG synthesis using a bIPG—a unidirectional iterative coupling approach.

FIG. 6. Reaction mechanism for deprotecting bIPGs represented by S033a-c.

FIG. 7. Example monomers for stepwise polymer synthesis containing a bIPG and a benzyl electrophile.

FIG. 8. Stepwise polymer synthesis using a bIPG—a bidirectional iterative coupling approach.

FIG. 9. Stepwise PEG synthesis using a bIPG—a bidirectional iterative coupling approach.

FIG. 10. Stepwise PEG synthesis using a bIPG—a chain doubling approach.

FIG. 11. Screening bIPGs for stepwise polymer synthesis.

FIG. 12. Synthesis of monomer S025.

FIG. 13. Synthesis of monomer S041.

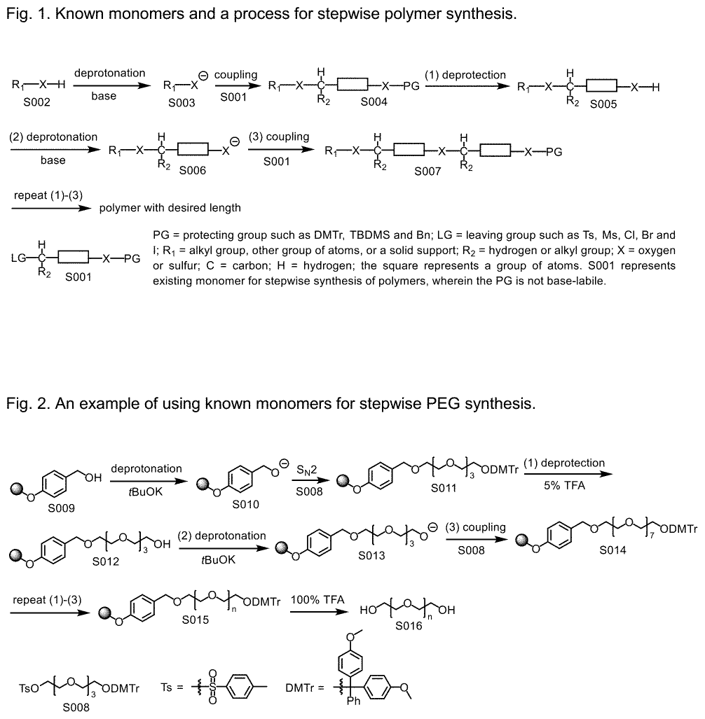

### DETAILED DESCRIPTION OF THE INVENTION

This invention is related to the use of bIPGs for stepwise synthesis of polymers. The reason for the use of stepwise synthesis instead of the less expensive polymerization method for polymer synthesis is usually the need of homogeneous polymers. Such polymers have been called sequence-defined polymers, monodisperse polymers or discrete polymers [Solleder et al 2017 Macromol Rapid Comm 38:1600711 doi:10.1002/marc.201600711, French et al 2009 Angew Chem Int Ed 48:1248 doi:10.1002/anie.200804623].

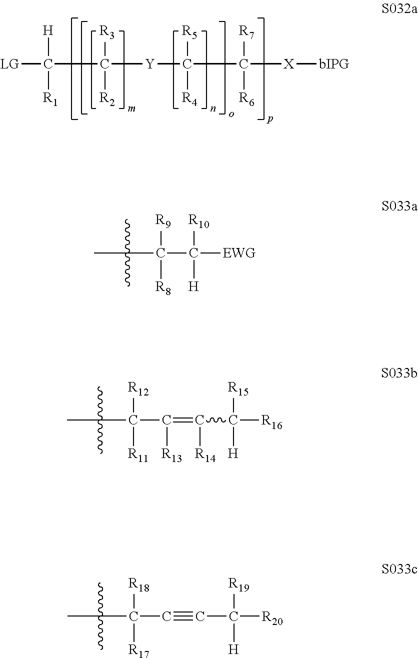

Some embodiments of the invention are related to the use of S032a as the monomer for stepwise polymer synthesis. The base-labile protecting group (bIPG) in S032a can be S033a-c. The subscripts m, n and p are positive integers, and o is zero or a positive integer. X and Y are oxygen or sulfur. R₁-R_₇ are independently hydrogen, alkyl group or substituted alkyl groups. The leaving group can be anything that can enable an S_{N}2 reaction under basic conditions. They include but not limited to sulfonates and halides. Sulfonates include but not limited to tosylate, benzenesulfonate, substituted benzene sulfonates, mesylate and triflate.

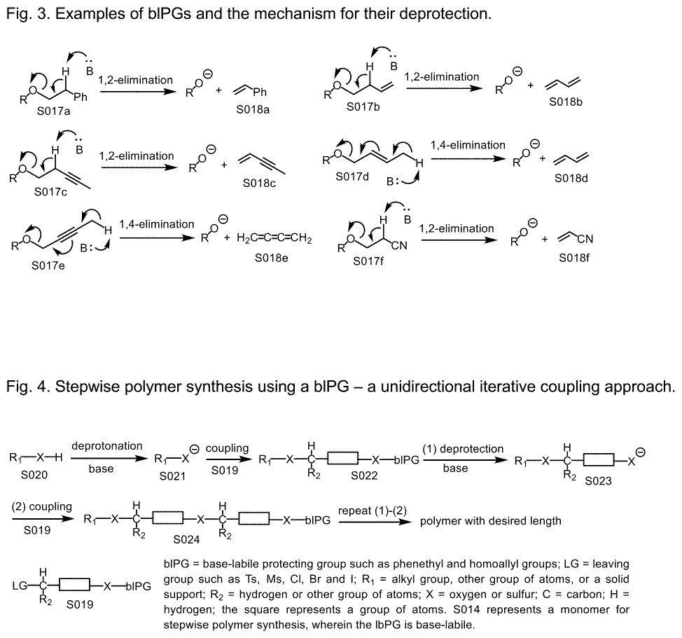

For the embodiments related to S032a with bIPG being S033a (S032a/S033a), R₈-R₁₀ are independently hydrogen, alkyl groups or substituted alkyl groups. EWG, which stands for electron withdrawing group, includes but not limited to phenyl group, substituted phenyl groups, other aryl groups including those with heteroaromatic groups, vinyl group, substituted vinyl group, prop-1-yn-1-yl group and substituted prop-1-yn-1-yl groups, sulfonato group, carboxylate group, cyano group, and dithiane group (in this case, R₁₀ and the carbon attached to it in the formula are part of EWG with the carbon attached to R₁₀ being carbon-2 of the dithiane). The general deprotection reaction mechanism is shown in FIG. 6 (S034a) wherein X is oxygen or sulfur, and R can be considered as an alkyl or substituted alkyl group. Examples of reaction mechanism for several specific protecting groups are given in FIG. 3 (S017a-c and S017f).

For the embodiments related to S032a/S033b, R₁₁-R₁₆ are independently hydrogen, alkyl groups or substituted alkyl groups. The general deprotection reaction mechanism is shown in FIG. 6 (S034b). An example reaction mechanism for a specific protecting group in these embodiments is given in FIG. 3 (S017d).

For the embodiments related to S032a/S033c, R₁₇-R₂₀ are independently hydrogen, alkyl groups or substituted alkyl groups. The general deprotection reaction mechanism is shown in FIG. 6 (S034c). An example reaction mechanism for a specific protecting group in these embodiments is given in FIG. 3 (S017e).

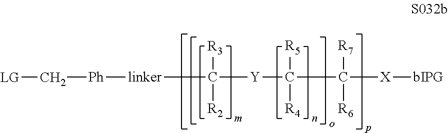

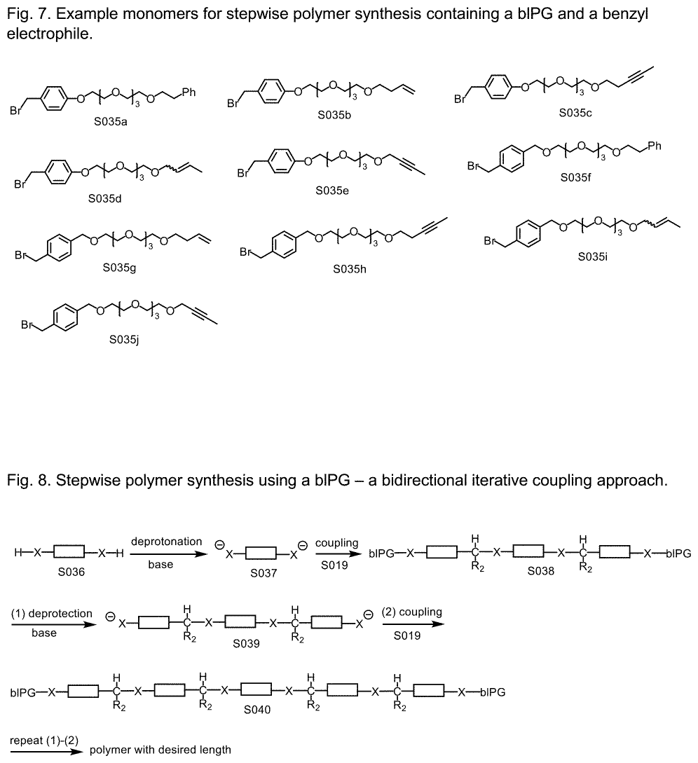

Some embodiments of the invention are related to the use of S032b as the monomer for stepwise polymer synthesis. The variables bIPG, LG, m, n, o, p, R₂, R₃, R₄, R₅, R₆, R₇, X and Y in S032b are the same as defined above for embodiments related to S032a. Ph is a phenyl group. The linker is one or more atoms that serve as a means for linking two portions of the molecule together. A difference between S032a and S032b is that the latter has a benzyl electrophile, which is usually more reactive in S_{N}2 reactions. In addition, benzyl electrophiles do not have the concern of β-elimination side reaction, which usually accompanies S_{N}2 reactions. Several example monomers (S035a-i) of the embodiments related to S032b are shown in FIG. 7. More examples can be readily derived from S032b. The synthesis of these monomers is straightforward for persons having ordinary skill in the art.

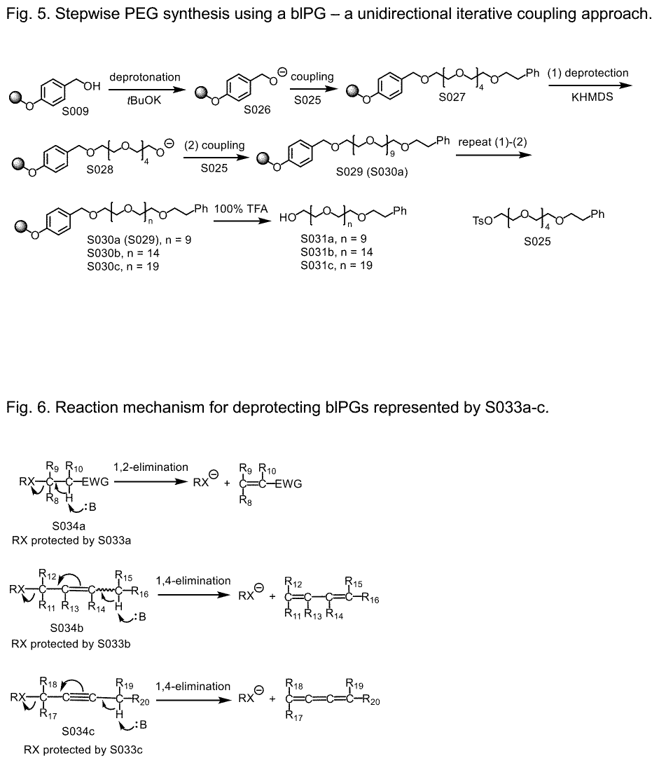

The approaches for stepwise polymer synthesis using S032a-b as the monomer include but not limited to unidirectional iterative coupling, bidirectional iterative coupling, chain doubling, and chain tripling. Many different versions of these approaches as well as other arrangements of the reactions are obvious to person having ordinary skill in the art, and they are not presented one by one here. A general procedure of some embodiments using a unidirectional iterative coupling approach has been shown in FIG. 4. The monomer used in FIG. 4 is S019, which is a more general version of S032a-b. Whether using S019 or S032a-b for the illustration, the process is the same. A specific example using a unidirectional iterative coupling approach for the synthesis of PEG has been shown in FIG. 5.

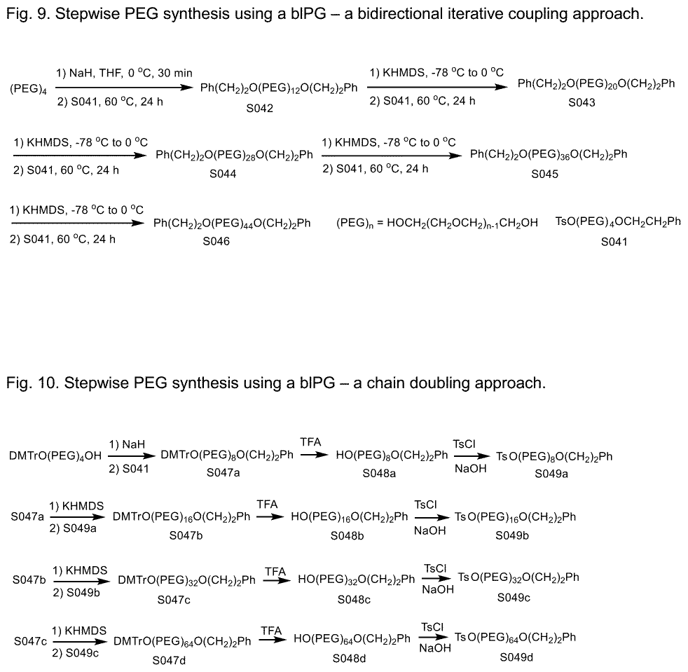

A general procedure of some embodiments using a bidirectional iterative coupling approach is shown in FIG. 8. Using this approach, the polymer grows faster, but the product is symmetric, which is not desirable in some applications. A specific example of using a bidirectional iterative coupling approach for PEG synthesis is shown in FIG. 9. The monomer S041 is used for the illustration.

An example of chain doubling approach involving the use of a bIPG for stepwise PEG synthesis is shown in FIG. 10. This example uses two orthogonal protecting groups, the base-labile phenethyl group and the acid-labile DMTr group. Using two orthogonal protecting groups is needed for the synthesis of asymmetric polymers using the chain doubling approach—here in the product S049d, the two ends of the molecule have different functionalities. The chain doubling approach also grows polymers faster. However, in each synthetic cycle, more steps are needed as compared with the unidirectional iterative coupling approach. The approach is beneficial for the synthesis of longer polymers. It may be beneficial to combine the chain doubling approach with the unidirectional iterative coupling approach for long polymer synthesis. For example, the (PEG)₂₀O(CH₂)₂Ph can be synthesized using unidirectional iterative coupling on a solid support or in solution, and then be converted to TsO(PEG)₂₀O(CH₂)₂Ph and (PEG)₂₀ODMTr. Coupling the two using the chain doubling approach can quickly reach DMTrO(PEG)₄₀O(CH₂)₂Ph.

The bIPG can also make the chain tripling stepwise polymer synthesis approach more efficient. Chain tripling approach grows polymer the fastest. However, in each synthetic cycle, multiple steps are needed and one of the step requires the desymmetrization of a symmetric polymer, which is challenging and gives low yield. Thus, chain tripling approach is not very useful for the synthesis of long polymers [French et al 2009 Angew Chem Int Ed 48:1248 doi:10.1002/anie.200804623].

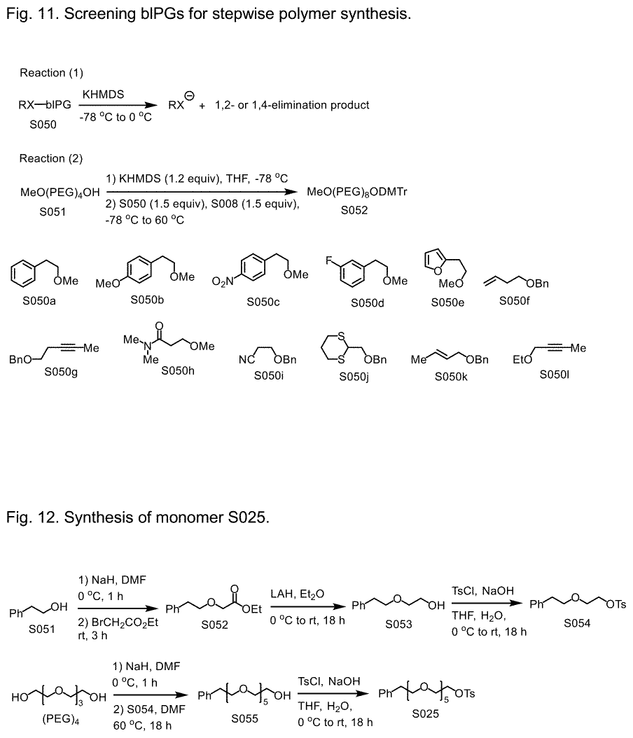

For a bIPG to be useful for stepwise polymer synthesis, it must meet two criteria [Mikesell et al 2021 Beilstein J Org Chem 17:2976 doi:10.3762/bjoc.17.207]. Criterion (i), it must be deprotectable by a base in the deprotection step. Criterion (ii), it must be stable under the basic conditions in the coupling step. Whether a bIPG meets the two criteria or not can be easily predicted using two simple reactions that mimic the deprotection and coupling reaction conditions planned for the stepwise polymer synthesis. For example, to screen suitable bIPGs planned to be used for the stepwise PEG synthesis shown in FIG. 9, model compounds with the general formula S050, which are usually readily accessible, can be subjected to the planned deprotection and coupling conditions in FIG. 9 as shown in FIG. 11. For reaction (1), if TLC analysis indicates that KHMDS (or other bases) can cause 1,2- or 1,4-elimination of a model compound, the bIPG in that compound is likely to meet criterion (i). For reaction (2), if TLC analysis indicates that product S052 can be formed from S051 and S008 while the model compound S050 is not affected by the basic conditions, the bIPG in the model compound is likely to meet criterion (ii). For the bIPGs in model compounds S050a-l, it has been found that all except for S050h meet both criteria. For S050h, it does not meet criterion (ii).

### EXPERIMENTAL EXAMPLES

### Example 1: Screening bIPGs for Stepwise PEG Synthesis—Testing if the Groups in S050a-l can be Removed Using KHDMS as the Base (FIG. 11, Reaction 1)

In an oven dried 25 mL flask, S050a-k or S050l (0.734 mmol, 1 equiv.) was dissolved in THF (4 mL). The solution was cooled to −78° C. KHMDS (1 M in THF, 1.468 mL, 1.468 mmol, 2 equiv.) was added via a syringe. The reaction mixture was stirred while warming to 0° C. gradually. After 2 h, TLC analyses were carried out. All compounds were found to be consumed. Thus, the base-labile protecting groups in them meet criterion (i), which is being labile under basic conditions [Mikesell et al 2021 Beilstein J Org Chem 17:2976 doi:10.3762/bjoc.17.207]. Compound S050a was also tested using the base tBuOK/LDA and found consumed under the conditions [Margot et al 1990 Tetrahedron 46:2425 doi:10.1016/50040-4020(01)82023-8, Margot et al 1990 Tetrahedron 46:2411 doi:10.1016/50040-4020(01)82022-6].

### Example 2: Screening bIPGs for Stepwise PEG Synthesis—Testing Stability of Protecting Groups Under the Basic Coupling Conditions (FIG. 11, Reaction 2)

Compounds S050, DMTrO(PEG)₄OTs (S008) [Khanal et al 2017 Chem Eur J 23:15133 doi:10.1002/chem.201703004] and MeO(PEG)₄OH (S051) were dried over P₂O₅ in a desiccator under vacuum for 2 days. Compound S051 (41 mg, 0.201 mmol, 1 equiv.) was dissolved in THF (200 μL) under nitrogen. The solution was cooled to −78° C., and KHMDS (0.241 mL, 0.241 mmol, 1 M in THF, 1.2 equiv.) was added dropwise via a syringe. After addition, the reaction flask was placed in an ice bath for ˜30 min. The mixture was then cooled to −78° C. The solution of S008 (195 mg, 0.301 mmol, 1.5 equiv.) and S050a-k or S050l(0.301 mmol, 1.5 equiv.) in THF (5004) was added via a cannula dropwise. The reaction mixture was warmed to rt gradually over ˜3 h. After stirring at rt for ˜30 min, the mixture was heated to 60° C. and stirred vigorously at the temperature for 24 h. TLC analyses were carried out to determine if the coupling reaction between S051 and S008 could proceed to form product S052 without the consumption of compound S050a-k or S050l. All the compounds except S050h were found to be able to survive the basic coupling reaction conditions. Thus, the base-labile protecting groups in them (except for S050h) meet criterion (ii), which is being stable under the basic coupling conditions required for the PEG synthesis.

### Example 3: Synthesis of S052 (FIG. 12)

The suspension of NaH (60% in mineral oil, 3.64 g, 82.8 mmol, 1.0 equiv.) in anhydrous DMF (150 mL) in a 2-neck round bottom flask under nitrogen was cooled on an ice bath. The solution of S051 (10.0 mL, 82.8 mmol, 1.0 equiv) in anhydrous DMF (250 mL) was added dropwise via a cannula over ˜1 h. After addition, the reaction mixture was stirred at 0° C. for ˜1 h. This gave the clear solution of NaO(CH₂)₂Ph. Ethyl bromoacetate (13.8 g, 82.8 mmol, 1.0 equiv) was dissolved in anhydrous DMF (100 mL). The solution of NaO(CH₂)₂Ph was added dropwise via a cannula. After addition, the mixture was stirred at 0° C. for 4 h, and the reaction was then quenched with EtOH. DMF was removed on a rotary evaporator under vacuum. The residue was partitioned between EtOAc (700 mL) and saturated NaCl (150 mL). The organic phase was washed with saturated NaCl (150 mL×3), dried over anhydrous MgSO₄, and filtered. The filtrate was evaporated to dryness under reduced pressure. The residue was dried under high vacuum, and purified with flash chromatography (SiO₂, EtOAc/hexanes 1:4) to give compound S052 (14.4 g, 83%) as a clear oil: TLC R_{f}=0.6 (SiO₂, hexanes/EtOAc 4:1); ¹H NMR (400 MHz, CDCl₃) δ 7.27-7.17 (m, 5H), 4.17 (d, J=8.0 Hz, 2H), 4.04 (s, 2H), 3.73 (t, J=8.0 Hz, 2H), 2.92 (t, J=8.0 Hz, 2H), 1.24 (t, J=8.0 Hz, 3H); ¹³C NMR (100 MHz, CDCl₃) δ 170.48, 138.58, 129.03, 128.53, 126.45, 72.83, 68.71, 61.03, 36.45, 14.53; HRMS (ESI) m/z: calcd for [M+Na]⁺ 231.0997; found, 231.0987.

### Example 4: Synthesis of S053 (FIG. 12)

Lithium aluminum hydride (LAH) (1.98 g, 51.8 mmol, 0.75 equiv.) was placed in a two neck round bottom flask and flushed with nitrogen. The flask was placed on an ice bath. Anhydrous Et₂O (75 mL) in another flask under nitrogen was added dropwise via a cannula. To the mixture, the solution of S052 (14.4 g, 69.1 mmol, 1.0 equiv) in anhydrous Et₂O (300 mL) was added dropwise via a cannula over ˜1 h. After addition, the reaction mixture was stirred at rt for 8 h. The reaction was quenched at 0° C. by sequential dropwise addition of water (1.98 mL), 15% NaOH solution (1.98 mL) and water (5.94 mL). The white solid was filtered off, and the filtrate was dried over anhydrous MgSO₄. The solution was evaporated to dryness under reduced pressure. The residue was purified with flash chromatography (SiO₂, EtOAc/hexanes 1:5) to give compound S053 (9.96 g, 86%) as a clear oil: TLC R_{f}=0.3 (SiO₂, hexanes/EtOAc 4:1); ¹H NMR (400 MHz, CDCl₃) δ 7.28-7.18 (m, 5H), 3.67 (t, J=8.0 Hz, 4H), 3.52 (t, J=4.0 Hz, 2H), 2.88 (t, J=8.0 Hz, 2H); ¹³C NMR (100 MHz, CDCl₃) δ 138.91, 128.55, 128.50, 126.44, 72.18, 61.96, 36.53; HRMS (ESI) m/z: calcd for [M+Na]⁺, 189.0892; found, 189.0881.

### Example 5: Synthesis of S054 (FIG. 12)

Compound S053 (5.7 g, 31.1 mmol, 1.0 equiv.) in THF (70 ml) in a round bottom flask was cooled on an ice bath. To the flask was added the solution of NaOH (12.45 g, 311 mmol, 10 equiv.) in water (70 ml). After the mixture was stirred at 0° C. for 1 h, p-toluene sulfonyl chloride (8.86 g, 46.6 mmol, 1.5 equiv.) in THF (140 mL) was added dropwise via a cannula over ˜1 h. After addition, the mixture was stirred for 18 h while warming to rt gradually. The mixture was partitioned between EtOAc (500 mL) and saturated NaCl (50 mL). The organic phase was washed with saturated NaCl (50 mL×3), dried over anhydrous MgSO₄ and filtered. The filtrate was evaporated to dryness under reduced pressure. The residue was purified with flash chromatography (SiO₂, EtOAc/hexanes 1:4) to give compound S054 (7.06 g, 98%) as a clear oil: TLC R_{f}=0.6 (SiO₂, hexanes/EtOAc 4:1); ¹H NMR (400 MHz, CDCl₃) δ 7.76-7.74 (d, 2H) 7.30-7.12 (m, 8H), 4.11 (t, J=4.0 Hz, 2H), 3.57 (m, 4H), 2.78 (t, J=8.0 Hz, 2H), 2.40 (s, 3H); ¹³C NMR (100 MHz, CDCl₃) δ 144.89, 138.74, 133.19, 129.94, 128.11, 126.41, 72.47, 69.49, 68.46, 36.41, 21.94; HRMS (ESI) m/z: calcd for [M+Na]⁺, 343.0980; found, 343.0967.

### Example 6: Synthesis of S055 (FIG. 12)

The suspension of NaH (60% in mineral oil, 0.98 g, 24.5 mmol, 1.2 equiv.) in anhydrous DMF (50 mL) in a 2-neck round bottom flask under nitrogen was cooled on an ice bath. The solution of tetraethylene glycol (PEG₄, 19.7 g, 17.5 mL, 204 mmol, 5.0 equiv.) in anhydrous DMF (150 mL) was added dropwise via a cannula over ˜1 h. The mixture was stirred at 0° C. for ˜1 h giving a clear solution of NaOPEG₄OH. The solution was warmed to rt and then heated to 60° C. Compound S054 (4.7 g, 20.4 mmol, 1.0 equiv.) in anhydrous DMF (50 mL) was added dropwise via a cannula over ˜3 h. After addition, the mixture was stirred at 60° C. for 8 h. The reaction was quenched with EtOH, and DMF was removed on a rotary evaporator under vacuum. The residue was partitioned between EtOAc (400 mL) and saturated NaCl (50 mL). The organic phase was washed with saturated NaCl (50 mL×3), dried over anhydrous MgSO₄ and filtered. The filtrate was evaporated to dryness under reduced pressure. The residue was purified with flash chromatography (SiO₂, EtOAc/hexanes 2:1) to give compound S055 (4.73 g, 68%) as a clear oil: TLC R_{f}=0.3 (SiO₂, hexanes/EtOAc 1:2); ¹H NMR (400 MHz, CDCl₃) δ 7.17-7.09 (m, 5H), 3.61-3.54 (m, 22H), 2.80 (t, J=8.0 Hz, 2H); ¹³C NMR (100 MHz, CDCl₃) δ 138.96, 129.02, 128.39, 126.23, 72.45, 70.82, 70.75, 70.43, 69.30, 36.47; HRMS (ESI) m/z: calcd for [M+Na]⁺, 365.1940; found, 365.1922.

### Example 7: Synthesis of S025 (FIG. 12)

Compound S055 (4.3 g, 12.5 mmol, 1.0 equiv.) in THF (30 mL) in a round bottom flask was cooled on an ice bath. NaOH (5.0 g, 125 mmol, 10 equiv.) in water (30 ml) was added. The mixture was stirred vigorously at 0° C. for 1 h. p-Toluene sulfonyl chloride (3.5 g, 18.8 mmol, 1.5 equiv.) in THF (60 mL) was added dropwise via a cannula over ˜1 h. After addition, the mixture was stirred for ˜18 h while warming to rt gradually. The mixture was partitioned between EtOAc (200 mL) and saturated NaCl (25 mL). The organic phase was washed with saturated NaCl (25 mL×3), dried over anhydrous MgSO₄, and filtered. The filtrate was evaporated to dryness under reduced pressure. The residue was purified with flash chromatography (SiO₂, EtOAc/hexanes 1:1) to give compound S025 (5.23 g, 92%) as a clear oil: TLC R_{f}=0.4 (SiO₂, hexanes/EtOAc 1:1); ¹H NMR (400 MHz, CDCl₃) δ 7.74-7.72 (d, 2H) 7.29-7.15 (m, 7H), 4.09 (t, J=4.0 Hz, 2H), 3.62-3.51 (m, 20H), 2.84 (t, J=8.0 Hz, 2H), 2.38 (s, 3H); ¹³C NMR (100 MHz, CDCl₃) δ 144.88, 139.02, 133.15, 129.95, 129.03, 128.08, 126.28, 72.50, 70.94, 70.48, 69.51, 68.88, 36.53, 21.93; HRMS (ESI) m/z: calcd for [M+Na]⁺, 519.2028; found, 519.2007.

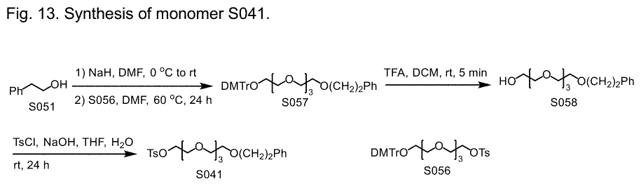

### Example 8: Synthesis of S057 (FIG. 13)

The suspension of NaH (60% in mineral oil, 716 mg, 17.9 mmol, 2.5 equiv.) in anhydrous DMF (25 mL) in a 2-neck round bottom flask under nitrogen was cooled on an ice bath. The solution of S051 (2.14 mL, 17.9 mmol, 2.5 equiv.) in anhydrous DMF (15 mL) was added dropwise via a cannula over ˜1 h. After addition, the reaction mixture was stirred at 0° C. for ˜1 h. The ice bath was removed. This gave the solution of NaO(CH₂)₂Ph. Compound S056 [Khanal et al 2017 Chem Eur J 23:15133 doi:10.1002/chem.201703004] (4.66 g; 7.17 mmol, 1 equiv.), which had been dried over P₂O₅ under high vacuum overnight, was dissolved in anhydrous DMF (15 mL). The solution was added to the solution of NaO(CH₂)₂Ph dropwise via a cannula. After addition, the mixture was stirred vigorously at 60° C. for 24 h. After cooling to rt, the reaction was quenched with EtOH. DMF was removed on a rotary evaporator under high vacuum. The residue was partitioned between EtOAc (250 mL) and 5% K₂CO₃ (100 mL). The organic phase was washed with 5% K₂CO₃ (100 mL×3), dried over anhydrous Na₂SO₄, and filtered. The filtrate was evaporated to dryness under reduced pressure and further dried under high vacuum. The residue was purified with flash chromatography (SiO₂, Et₃N/hexanes 1:9) to give compound S057 (4.02 g, 96%) as a yellow oil: TLC R_{f}=0.3 (SiO₂, hexanes/EtOAc 3:1); ¹H NMR (400 MHz, CDCl₃) δ 7.49-7.47 (d, 2H), 7.37-7.35 (d, 4H), 7.29-7.18 (m, 8H), 6.83-6.80 (m, 4H), 2.76-2.69 (m, 8H), 3.74 (s, 6H), 3.68-3.59 (m, 16H), 3.25-3.23 (t, 2H), 2.91-2.87 (t, 2H); ¹³C NMR (100 MHz, CDCl₃) δ 158.5, 145.3, 139.1, 136.5, 130.3, 129.1, 128.5, 128.4, 127.9, 126.8, 126.3, 113.2, 86.2, 72.6, 71.0, 70.5, 63.5, 55.5, 36.6; HRMS (ESI) calcd for C₃₇H₄₃O₇Na [M+Na]⁺623.2985, found 623.2971.

### Example 9: Synthesis of S058 (FIG. 13)

Compound S057 (2.17 g, 3.62 mmol, 1 equiv.) was dissolved in dry DCM (10 mL). To the solution was added TFA (433 μL, 3.62 mmol, 1 equiv.). The reaction mixture was stirred vigorously. After ˜5 mins, TLC indicated that compound S057 was consumed. The reaction was quenched with solid NaOH and a small volume of water until pH ˜9. The mixture was then partitioned between DCM (total about 200 mL) and brine (75 mL). The aqueous phase was washed with DCM (100 mL×3). The combined organic phase was dried over anhydrous Na₂SO₄, and filtered. The filtrate was evaporated to dryness, and the residue was purified with flash chromatography (SiO₂, EtOAc) to give compound S058 (568 mg, 77%) as a yellow oil: TLC R_{f}=0.10 (SiO₂, hexanes/EtOAc 1:3); ¹H NMR (400 MHz, CDCl₃) δ 7.25-7.10 (m, 5H), 3.67-3.64 (t, 2H), 3.62-3.53 (m, 16H), 2.87-2.83 (t, 2H); ¹³C NMR (100 MHz, CDCl₃) δ 138.9, 129.1, 128.5, 126.3, 72.8, 70.8, 70.5, 91.9, 36.5; HRMS (ESI) calcd for C₁₆H₂₆O₅H [M+H]⁺ 299.1859, found 299.1847; C₁₆H₂₆O₅Na [M+Na]⁺321.1678, found 321.1662.

### Example 10: Synthesis of S041 (FIG. 13)

The solutions of S058 (9.22 g, 46.5 mmol, 1 equiv.) in THF (50 mL) and NaOH powder (22.3 g, 557 mmol, 12 equiv.) in water (50 mL) were combined and stirred at 0° C. for 5 min. The solution of TsCI (26.5 g, 139.5 mmol, 3 equiv.) in THF (50 mL) was added dropwise over 10 min while the reaction mixture was stirred at 0° C. After addition, stirring was continued while the temperature was raised to rt gradually. The progress of the reaction was monitored by TLC, and complete reaction was observed within 24 h. The mixture was partitioned between 5% Na₂CO₃ (300 mL) and EtOAc (500 mL). The aqueous phase was extracted with EtOAc (200 mL×3). The combined organic phase was dried over anhydrous Na₂SO₄ and filtered. Volatiles were removed under reduced pressure, and the residue was further dried under vacuum from an oil pump. Compound S041 (12.7 g, 60%) was obtained as a colorless oil after flash chromatography purification (SiO₂, hexanes/EtOAc 1:0 to 2:1): TLC R_{f}=0.30 (SiO₂, hexanes/EtOAc 1:1); ¹H NMR (400 MHz, CDCl₃) δ 7.78-7.76 (d, 2H), 7.32-7.30 (d, 2H), 7.27-7.16 (m, 5H), 4.14-4.12 (t, 2H), 3.68-3.59 (m, 16H), 2.89-2.86 (t, 2H), 2.42 (s, 3H); ¹³C NMR (100 MHz, CDCl₃) δ 144.9, 139.0, 133.2, 129.9, 129.1, 128.5, 128.1, 126.3, 72.5, 70.9, 70.8, 70.7, 70.5, 69.5, 68.9, 36.5, 21.9; HRMS (ESI) calcd for C₂₃H₃₁O₇SH [M+H]⁺ 453.1942, found 453.1953; C₂₃H₃₁O₇SNH₄ [M+NH₄]⁺ 470.2207, found 470.2216; C₂₃H₃₁O₇SNa [M+Na]⁺ 474.1761, found 475.1775.

### Example 11: Stepwise PEG Synthesis Using the Unidirectional Iterative Coupling Approach (FIG. 5)

Automated solid phase synthesis: The CBS Bio CS136X peptide synthesizer was modified for the automated synthesis. The synthesizer has two measuring vessels called MVA and MVB, which use sensors to determine the volume of solutions or solvents to be delivered to the reaction vessel (RV). MVA is used to measure solutions or solvents that need to be kept anhydrous. MVB is used to measure solutions or solvents that contain water or acids, or to measure solutions or solvents that do not need to be kept anhydrous. To meet the needs of the project, several reagent or solvent bottles connected to MVA were changed to connect to MVB, and the software was modified to accommodate the modification. In addition, the argon going into the synthesizer was dried via molecular sieve in a drying tube, and the gas venting lines of the synthesizer were connected to a drying tube filled with Drierite before reaching to air. An example synthesis is given. To prepare for the synthesis, the Wang resin S009 (12, 1.0 g, 0.9 mmol/g loading, 0.9 mmol) was loaded into a 20 ml RV. Dry THF (15 ml) was delivered to the RV, and the resin was allowed to swell at rt for 10 min. Mixing of the resin and solvent was achieved by rotating the RV 180° back and forth, which is the mixing mechanism of the synthesizer. After draining, the resin was washed with anhydrous solvents. The washing scheme of sequential THF, DMF, DMSO and NMP washes with 10 min waiting and five repetitions was used. For converting S009 to S027, KHMDS (or tBuOK) in THF (0.25 M, 15 ml, 3.75 mmol, 4.1 equiv.) was delivered to RV for deprotonation. After mixing at rt for 5 min, the solution was drained. The deprotonation was repeated one time. After draining, the resin was washed with anhydrous DMF two times. The solution of monomer S025 (0.5 M in DMF, 15 ml, 7.5 mmol, 8.33 equiv.) was delivered into RV, and the materials were mixed at rt for 6 h. The solution was drained, and the resin was washed with THF (10 mL×2), THF/H₂O (v/v 1:1, 15 mL×5); THF (10 mL×3); DMF (10 mL×3); DMSO (10 mL×3). For converting S027 to S029 (i.e. S030a), S030a to S030b, and S030b to S030c, the same conditions for converting S009 to S027 were used except that for converting S030a to S030b, and S030b to S030c, tBuOK could not serve as an alternative base, and KHMDS was used.

Cleavage of PEG from resin: To the resin (50 mg), extensively washed as described above and dried, in a 1.5 mL centrifuge tube was added TFA (300 μL). The mixture was shaken at rt for 2 h. The tube was spun shortly to bring down liquids to the bottom, and the supernatant was transferred to another 1.5 mL tube. The resin was washed with TFA (50 μL×2) and THF (50 μL×3). The supernatant and the washes were combined. Volatiles were evaporated under vacuum. To the residue was added water (100 μL). The tube was vortexed and centrifuged. The supernatant was transferred to another 1.5 mL tube. The volatiles were evaporated under vacuum. The residue was dissolved in THF (100 μL), vortexed and centrifuged. The supernatant was transferred to another 1.5 mL tube, and the PEG product was obtained by evaporating THF, or alternatively, by precipitating from the THF solution with Et₂O (200 μL).

S031a: ¹H NMR (400 MHz, CDCl₃) δ 7.22-7.14 (m, 5H), 3.58 (m, 42H), 2.86 (t, J=8.0 Hz, 2H); ¹³C NMR (100 MHz, CDCl₃) δ 138.90, 129.19, 128.31, 126.49, 72.69, 70.13, 36.41. HRMS (ESI) m/z: calcd for [M+NH₄]⁺ 580.37, found 580.42.

S031b: ¹H NMR (400 MHz, CDCl₃) δ 7.27-7.13 (m, 5H), 3.58 (m, 62H), 2.85 (t, J=8.0 Hz, 2H); ¹³C NMR (100 MHz, CDCl₃) δ 138.88, 129.03, 128.47, 126.28, 72.68, 69.90, 61.19, 36.45. HRMS (ESI) m/z: calcd for [M+NH₄]⁺ 800.50, found 800.50.

S031c: ¹H NMR (400 MHz, CDCl₃) δ 7.27-7.13 (m, 5H), 3.60 (m, 82H), 2.86 (t, J=8.0 Hz, 2H); ¹³C NMR (100 MHz, CDCl₃) δ 138.97, 129.04, 128.46, 126.31, 72.68, 70.67, 61.40, 36.52. HRMS (ESI) m/z: calcd for [M+NH₄]⁺ 1020.63, found 1020.58.

### Example 12: Synthesis of S042 (FIG. 9)

Compound S041 (2.19 g, 4.83 mmol, 2.5 equiv.) was dried over P₂O₅ under vacuum in a desiccator overnight. A suspension of NaH (60% in mineral oil, 193 mg, 4.83 mmol, 2.5 equiv.) in dry THF (5 mL) under nitrogen was cooled on an ice bath. The solution of (PEG)₄ (333 μL, 1.93 mmol, 1 equiv.) in dry THF (10 mL) was added via a cannula dropwise over ˜20 min. After addition, the reaction was allowed to proceed for ˜30 min. The ice bath was removed, and compound S041 in THF (10 mL) was added via a cannula dropwise over ˜10 min. After addition, the mixture was stirred vigorously at 60° C. for 24 h. The reaction was quenched with EtOH. THF was removed under reduced pressure. The residue was partitioned between DCM (100 mL) and saturated NH₄Cl (50 mL). The aqueous phase was washed with DCM (100 mL×3). The combined organic phase was dried over anhydrous Na₂SO₄ and filtered. The filtrate was evaporated to dryness, and compound S042 was purified with flash chromatography (SiO₂, EtOAc/MeOH 100:0 to 100:3) to give a colorless oil (1.4 g, 97%): TLC R_{f}=0.50 (SiO₂, DCM/Et₂O/MeOH 5:1:1); ¹H NMR (400 MHz, CDCl₃) δ 7.24-7.712 (m, 10H), 3.64-3.55 (m 51H), 2.87-2.83 (t, 4H); ¹³C NMR (100 MHz, CDCl₃) δ 133.9, 129.02, 128.44, 126.28, 72.52, 70.80, 70.90, 36.54 HRMS (ESI) calcd for C₄₀H₆₆O₁₃Na [M+Na]⁺777.4401, found 777.4436; C₄₀H₆₆O₁₃Na₂ [M+2Na]²⁺400.2150, found 400.2112.

### Example 13: Synthesis of S043 (FIG. 9)

Compounds S041 and S042 were dried over P₂O₅ in a desiccator under vacuum for 2 days. Compound S042 (1.3 g, 1.8 mmol, 1 equiv.) was dissolved in dry THF (5 mL) under nitrogen. The solution was cooled to −78° C., and KHMDS (4.6 mL, 1 M in THF, 2.5 equiv.) was added dropwise via a syringe. After addition, the reaction flask was placed in an ice bath for ˜3 h. TLC analysis indicated that both S042 and Ph(CH₂)₂O(PEG)₁₂ were not in the reaction mixture. The mixture was then cooled to −78° C. for ˜10 min, and the solution of S041 (3.8 g, 8.3 mmol, 4.5 equiv.) in THF (10 mL) was added dropwise via a cannula over ˜10 min. The reaction mixture was allowed to warm up to room temperature gradually over a period of ˜3 h. After stirring at room temperature for ˜30 min, the mixture was heated to 60° C. and stirred vigorously at the temperature for 24 h. THF was removed under reduced pressure. The residue was partitioned between DCM (100 mL) and saturated NH₄Cl (20 mL). The aqueous phase was washed with DCM (100 mL×3). The combined organic phase was dried over anhydrous Na₂SO₄ and filtered. Flash chromatography (SiO₂, EtOAc to DCM/Et₂O/MeOH 100:8:4) gave compound S043 (1.765 g, 86%) as a yellow waxy solid: TLC R_{f}=0.40 (SiO₂, DCM/Et₂O/MeOH 10:1:1); ¹H NMR (400 MHz, CDCl₃) δ 7.28-7.15 (m, 10H), 3.67-3.57 (m 81H), 2.90-2.88 (t, 4H); ¹³C NMR (100 MHz, CDCl₃) δ 138.97, 129.08, 128.50, 126.34, 72.60, 70.80, 70.50, 36.55; HRMS (ESI) calcd for C₅₆H₉₈O₂₁Na [M+Na]⁺ 1129.6499, found 1129.6533; C₅₆H₉₈O₂₁H₂ [M+2H]²⁺554.3379, found 554.3390.

### Example 14: Synthesis of S044 (FIG. 9)

S044 was synthesized using the procedure for the synthesis of S043. Compound S043 (1.77 g, 1.59 mmol, 1 equiv.) in THF (10 mL), KHMDS (3.39 mL, 1 M in THF, 2.2 equiv.), and S041 (3.24 g, 7.15 mmol, 4.5 equiv.) in THF (10 mL) gave the crude product, which was subjected to aqueous workup and chromatography purification as describe for S043. Compound S044 (1.6 g, 70%) was obtained as a yellow waxy solid: TLC R_{f}=0.40 (SiO₂, DCM/Et₂O/MeOH 10:1:1); ¹H NMR (400 MHz, CDCl₃) δ 7.27-7.15 (m, 10H), 3.67-3.56 (m 116H), 2.89-2.85 (t, 4H); ¹³C NMR (100 MHz, CDCl₃) δ 139.02, 129.04, 128.46, 126.30, 72.54, 70.80, 36.56; HRMS (ESI) calcd for C₇₄H₁₃₄O₃₀Na [M+Na]⁺ 1481.8596, found 1481.8571; C₇₄H₁₃₄O₃₀Na₂[M+2Na]²⁺ 752.4247, found 752.4247; C₇₄H₁₃₄O₃₀H₃ [M+3H]³⁺ 487.2977, found 487.2971.

### Example 15: Synthesis of S045 (FIG. 9)

S045 was synthesized using the procedure for the synthesis of S043. Compound S044 (1.375 g, 0.942 mmol, 1 equiv.) in THF (10 mL), KHMDS (2.4 mL, 1 M in THF, 2.5 equiv.), and S041 (1.7 g, 3.8 mmol, 4 equiv.) in THF (10 mL) gave the crude product, which was subjected to aqueous workup and chromatography purification as describe for S043. Compound S045 (436 mg, 25%) was obtained as a yellow waxy solid: TLC R_{f}=0.40 (SiO₂, DCM/Et₂O/MeOH 10:1:1); ¹H NMR (400 MHz, CDCl₃) δ 7.25-7.15 (m, 10H), 3.65-3.59 (m 148H), 2.87-2.83 (t, 4H); ¹³C NMR (100 MHz, CDCl₃) δ 139.00, 129.02, 128.44, 126.29, 72.53, 70.79, 70.49, 36.54; HRMS (ESI) calcd for C₈₈H₁₆₂O₃₇N₂H₈ [M+2NH₄]²⁺ 923.5742, found 923.5701; C₈₈H₁₆₂O₃₇N₃H₁₂ [M+3NH₄]³⁺ 621.7276, found 621.7269.

### Example 16: Synthesis of S046 (FIG. 9)

S046 was synthesized using the procedure for the synthesis of S043. Compound S045 (386 mg, 0.241 mmol, 1 equiv.) in THF (10 mL), KHMDS (0.532 mL, 1 M in THF, 2.5 equiv.), and S041 (436 mg, 0.964 mmol, 4 equiv.) in THF (10 mL) gave the crude product, which was subjected to aqueous workup and chromatography purification as describe for S043. Compound S046 (199 mg, 43%) was obtained as a yellow waxy solid: TLC, R_{f}=0.50 (SiO₂, DCM/Et₂O/MeOH 6:1:1); ¹H NMR (400 MHz, CDCl₃) δ 7.23-7.13 (m, 10H), 3.76-3.38 (m 179H), 2.85-2.81 (t, 4H); ¹³C NMR (100 MHz, CDCl₃) δ 138.95, 129.02, 128.44, 126.29, 72.53, 70.77, 70.47, 36.52; HRMS (ESI) calcd for C₁₀₄H₁₉₄O₄₅N₂H₈[M+2NH₄]²⁺ 1099.6790, found 1099.6711; C₁₀₄H₁₉₄O₄₅N₃H₁₂ [M+3NH₄]³⁺ 739.1308, found 739.1266; C₁₀₄H₁₉₄O₄₅N₄H₁₆ [M+4NH₄]⁴⁺ 558.8663, found 558.8548.

1. A process for stepwise polymer synthesis using one or more monomers containing a base-labile protecting group at one end and a leaving group at the other, wherein said process comprises at least a deprotection step under basic conditions to remove said base-labile protecting group and a coupling step using one of said monomers also under basic conditions to elongate the polymer, and said monomers are defined by (I) 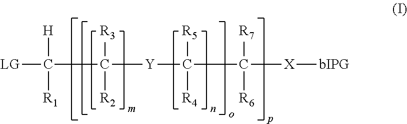
wherein m and n are independently a positive integer; p is a positive integer if o is a positive integer or an integer larger than two if o is zero; R₁, R₂, R₃, R₄, R₅, R₆ and R₇ are independently a hydrogen, alkyl group or substituted alkyl group; X and Y are independently an oxygen or sulfur atom; LG is a leaving group; and bIPG is said base-labile protecting group that is deprotectable using one or more bases in said deprotection step and is stable under the basic conditions in said coupling step, and is defined
by (II₁) 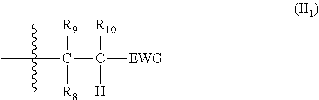
wherein R₈, R₉ and R₁₀ are independently a hydrogen, alkyl group or substituted alkyl group; and EWG (in some cases, R₁₀ is part of the EWG) is an electron withdrawing group that enables the hydrogen in II₁ acidic enough for deprotecting said bIPG in said deprotection step but not so acidic that said bIPG is unstable in said coupling step,
by (II₂) 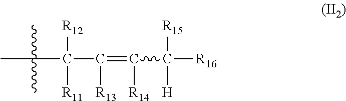
wherein R₁₁, R₁₂, R₁₃, R₁₄ and R₁₅ are independently a hydrogen, alkyl group or substituted alkyl group,
or
by (II₃) 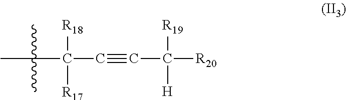
wherein R₁₇, R₁₈, R₁₉ and R₂₀ are independently a hydrogen, alkyl group or substituted alkyl group.

2. The process of claim 1, wherein o is zero.

3. The process of claim 1, wherein m, n and p are the integer one, and o is a positive integer.

4. The process of claim 1, wherein m, n and p are the integer one, and o is an integer larger than two.

5. The process of claim 1, wherein m, n and p are the integer one, o is a positive integer, R₁, R₃, R₄, R₅ and R₇ are hydrogen, and R₂ and R₆ are independently hydrogen, methyl group, ethyl group, propyl group, isopropyl group, butyl group or isobutyl group.

6. The process of claim 1, wherein m, n and p are the integer one, o is an integer larger than two, R₁, R₃, R₄, R₅ and R₇ are hydrogen, and R₂ and R₆ are independently hydrogen, methyl group, ethyl group, propyl group, isopropyl group, butyl group or isobutyl group.

7. The process of claim 1, wherein said bIPG is (II₁).

8. The process of claim 1, wherein said bIPG is (II₁) with its EWG being phenyl group, 4-fluoropheny group, 4-methylphenyl group, 4-nitrophenyl group, 4-methoxyphenyl group, sulfonato group, carboxylate group, vinyl group or prop-1-yn-1-yl group.

9. The process of claim 1, wherein said bIPG is (II₁), m, n and p are the integer one, o is a positive integer, R₁, R₃, R₄, R₅ and R₇ are hydrogen, and R₂ and R₆ are independently hydrogen, methyl group, ethyl group, isopropyl group, butyl group or isobutyl group.

10. The process of claim 1, wherein said bIPG is (II₂).

11. The process of claim 1, wherein said bIPG is (II₂), m, n and p are the integer one, o is a positive integer, R₁, R₃, R₄, R₅ and R₇ are hydrogen, and R₂ and R₆ are independently hydrogen, methyl group, ethyl group, propyl group, isopropyl group, butyl group or isobutyl group.

12. The process of claim 1, wherein said bIPG is (II₃).

13. The process of claim 1, wherein said bIPG is (II₃), m, n and p are the integer one, o is a positive integer, R₁, R₃, R₄, R₅ and R₇ are hydrogen, and R₂ and R₆ are independently hydrogen, methyl group, ethyl group, propyl group, isopropyl group, butyl group or isobutyl group.

14. The process of claim 1, wherein (I) is selected from (III₁-III₅): 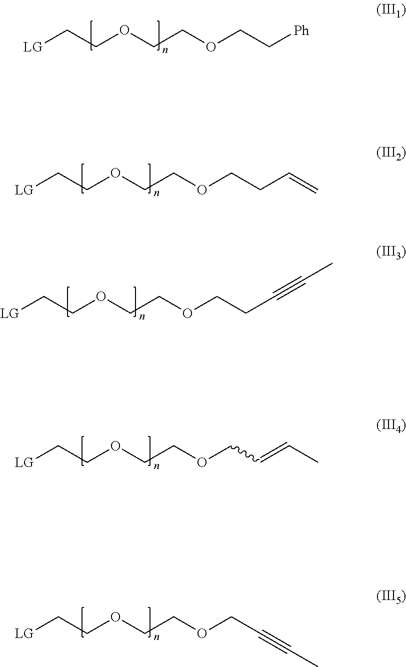
wherein n is an integer larger than 1, and LG is a leaving group selected from tosylate, benzenesulfonate, 4-nitrobenzenesulfonate, 4-fluorobenzenesulfonate, mesylate, triflate, chloride, bromide and iodide.

15. Compounds having the formula (I),
wherein m, n and p are independently a positive integer; o is an integer larger than two;
R₁, R₂, R₃, R₄, R₅, R₆ and R₇ are independently a hydrogen, alkyl group or substituted alkyl group;
X and Y are independently an oxygen or sulfur atom; LG is a leaving group; and bIPG is a base-labile protecting group defined
by (II₁), wherein R₈, R₉ and R₁₀ are independently a hydrogen, alkyl group or substituted alkyl group, and EWG is an electron withdrawing group limited to
alkenyl groups with less than eight carbons; alkynyl groups with three to eight carbons; phenyl group; furyl group; N-alkyl pyrrolyl group; substituted phenyl groups, furyl groups and N-alkyl pyrrolyl groups with substituent or substituents being halogen atom, nitro group, cyano group, alkyl group with less than eight carbons, alkoxyl group with its alkyl moiety having less than eight carbons, and dialkyl amino group with its alkyl moiety having less than eight carbons; bis(alkylthio) groups; and S,S′-(alkane-diyl) groups;
by (II₂), wherein R₁₁, R₁₂, R₁₃, R₁₄ and Rig are independently a hydrogen or alkyl group having less than five carbons;
or
by (II₃), wherein R₁₇, R₁₈, R₁₉ and R₂₀ are independently a hydrogen or alkyl group having less than five carbons.

16. Compounds of claim 15, wherein (I) is selected from (III₁-III₅) with n being an integer larger than 2, and LG is a leaving group selected from tosylate, benzenesulfonate, 4-nitrobenzenesulfonate, 4-fluorobenzenesulfonate, mesylate, triflate, chloride, bromide and iodide.

17. A process for stepwise polymer synthesis using one or more monomers containing a base-labile protecting group at one end and a leaving group at the other, wherein said process comprises at least a deprotection step under basic conditions to remove said base-labile protecting group and a coupling step using one of said monomers also under basic conditions to elongate the polymer, and said monomers are defined by (IV) 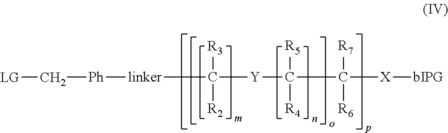
wherein m and n are independently a positive integer; p is a positive integer if o is a positive integer or an integer larger than two if o is zero; R₂, R₃, R₄, R₅, R₆ and R₇ are independently a hydrogen, alkyl group or substituted alkyl group; X and Y are independently an oxygen or sulfur atom; LG is a leaving group; linker is one or more atoms that connect the two portions of the molecule together; and bIPG is said base-labile protecting group that is deprotectable using one or more bases in said deprotection step and is stable under the basic conditions in said coupling step, and is defined
by (II₁), wherein R₈, R₉ and R₁₀ are independently a hydrogen, alkyl group or substituted alkyl group; and EWG (in some cases, R₁₀ is part of the EWG) is an electron withdrawing group that enables the hydrogen in II₁ acidic enough for deprotecting said bIPG in said deprotection step but not so acidic that said bIPG is unstable in said coupling step,
by (II₂), wherein R₁₁, R₁₂, R₁₃, R₁₄ and R₁₅ are independently a hydrogen, alkyl group or substituted alkyl group,
or
by (II₃), wherein R₁₇, R₁₈, R₁₉ and R₂₉ are independently a hydrogen, alkyl group or substituted alkyl group.

18. The process of claim 17, wherein (IV) is selected from (V₁-V₅): 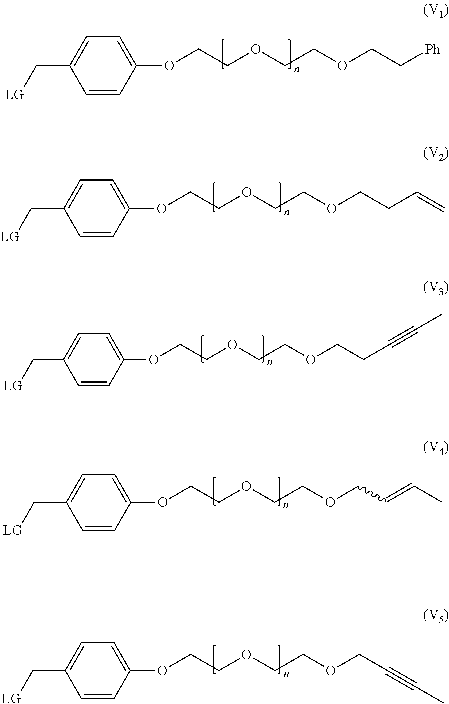
wherein n is an integer larger than 1, and LG is a leaving group selected from tosylate, benzenesulfonate, 4-nitrobenzenesulfonate, 4-fluorobenzenesulfonate, mesylate, triflate, chloride, bromide and iodide.

19. The process of claim 17, wherein (IV) is selected from (VI₁-VI₅): 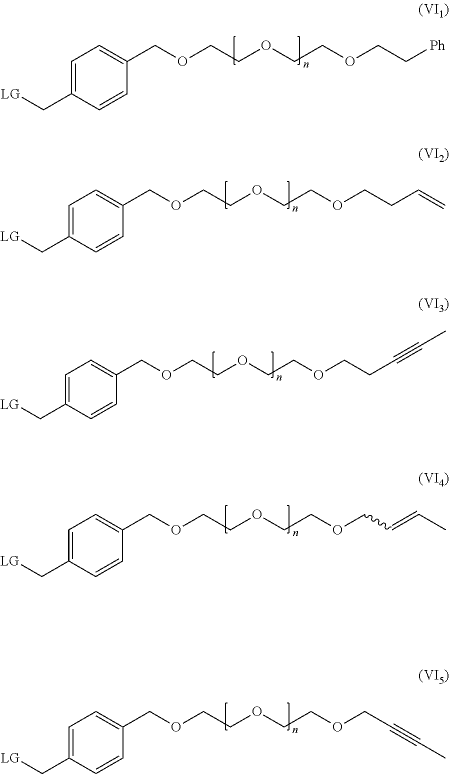
wherein n is an integer larger than 2, and LG is a leaving group selected from tosylate, benzenesulfonate, 4-nitrobenzenesulfonate, 4-fluorobenzenesulfonate, mesylate, triflate, chloride, bromide and iodide.
